SEQUENCE LISTING (20 sequences)

SEQ ID NO: 1 - DNA, 29 bp, Artificial
  primer
atggattctagaacagttggtatattagg

SEQ ID NO: 2 - DNA, 30 bp, Artificial
  primer
ttacttgttttctagataagcttcgtaacc

SEQ ID NO: 3 - DNA, 20 bp, Artificial
  primer
tacgctcgttccaagggatt

SEQ ID NO: 4 - DNA, 20 bp, Artificial
  primer
tggtgcttggttggttccat

SEQ ID NO: 5 - DNA, 20 bp, Artificial
  primer
cagcagcacgggtctgtcta

SEQ ID NO: 6 - DNA, 26 bp, Artificial
  primer
cggtttcttattcaattgctctaaca

SEQ ID NO: 7 - DNA, 20 bp, Artificial
  primer
tgccaagcaaccaacttacg

SEQ ID NO: 8 - DNA, 20 bp, Artificial
  primer
cggtggtcttaccatgcttc

SEQ ID NO: 9 - DNA, 17 bp, Artificial
  primer
gcgcatgaacccaacca

SEQ ID NO: 10 - DNA, 17 bp, Artificial
  primer
cgaccgcgtgaccgtta

SEQ ID NO: 11 - DNA, 22 bp, Artificial
  primer
gacatgatgccaaaagtggaaa

SEQ ID NO: 12 - DNA, 22 bp, Artificial
  primer
ccaccatcgacttggatattca

SEQ ID NO: 13 - DNA, 17 bp, Artificial
  primer
tgccggtgtcccaaaaa

SEQ ID NO: 14 - DNA, 19 bp, Artificial
  primer
tgcagctctcttggcatcc

SEQ ID NO: 15 - DNA, 19 bp, Artificial
  primer
ggcgcagatgacgagaaga

SEQ ID NO: 16 - DNA, 23 bp, Artificial
  primer
cagagcccggtacgtatctatga

SEQ ID NO: 17 - DNA, 22 bp, Artificial
  primer
ccaactggtttcaagatcggta

SEQ ID NO: 18 - DNA, 20 bp, Artificial
  primer
tccacagtggccaattgtga

SEQ ID NO: 19 - DNA, 22 bp, Artificial
  primer
cggcaaatacaggtgatgaaac

SEQ ID NO: 20 - DNA, 23 bp, Artificial
  primer
tcctccaacgagatgactttttc

(19) United States
(12) Patent Application Publication
(10) Pub. No.: US 2012/0149081 A1
(43) Pub. Date: Jun. 14, 2012

(54) METHOD FOR FERMENTATION CULTURE IN MEDIUM CONTAINING XYLOSE

(75) Inventors: Noriko SHISA, Nisshin-shi (JP); Kenro TOKUHIRO, Aichi-gun (JP); Satoshi KATAHIRA, Nagoya-shi (JP); Risa NAGURA, Toyota-shi (JP)

(73) Assignee: TOYOTA JIDOSHA KABUSHIKI KAISHA, Toyota-shi (JP)

(21) Appl. No.: 13/314,981

(22) Filed: Dec. 8, 2011

(30) Foreign Application Priority Data
Dec. 9, 2010 (JP) 2010-274415

(51) Int. Cl. C12P 7/06

(52) U.S. Cl. 435/161

(57) ABSTRACT

The xylose-metabolizing ability and particularly the xylose incorporation rate, of yeast to which xylose-metabolizing ability has been imparted are significantly improved. The method according to the present invention comprises the steps of: culturing yeast having xylose-metabolizing ability in a xylose-containing medium in which the concentration of at least one amino acid selected from the group consisting of asparagine (Asn), serine (Ser), tyrosine (Tyr), threonine (Thr), and histidine (His) is increased; and recovering alcohol from the medium.

### BACKGROUND OF THE INVENTION

1. Field of the Invention

The present invention relates to a fermentation culture method using xylose as a carbon source.

2. Background Art

There are high expectations for alternative energy resources to petroleum, such as ethanol, which are produced from woody biomass. Specifically, woody biomass is effectively used as a raw material for useful alcohols such as ethanol or organic acid. Woody biomass is mainly composed of cellulose, hemicellulose, and lignin. For production of liquid fuel such as ethanol from woody biomass, cellulose or hemicellulose is hydrolyzed (saccharified) to its constitutive monosaccharides, and then the monosaccharides are converted to ethanol by fermentation. Cellulose is mainly composed of glucose and hemicellulose is mainly composed of arabinose and xylose. Therefore, when ethanol is produced using woody biomass, it is desirable to effectively use not only glucose, but also xylose as a substrate for fermentation.

However, with yeast, which is generally used for ethanol production, xylose (pentose) cannot be used as a substrate. Accordingly, a technology of using yeast to which xylose-metabolizing ability has been imparted so that xylose may be used as a substrate is disclosed in Patent Documents 1 and 2 as well as Non-Patent Document 1. However, yeast to which xylose-metabolizing ability has been imparted has ethanol-fermenting ability due to the use of glucose as a substrate. Hence, the technology is problematic in that when a medium contains glucose and xylose, glucose fermentation preferentially proceeds and thus xylose metabolization is delayed.

Also, Non-Patent Document 1 discloses the results of examining recombinant yeast (S. cerevisiae), into which a xylose.reductase gene and a xylitol.dehydrogenase gene have been introduced, for the growth rates and the like resulting from medium composition employed. More specifically, the document discloses the results of examining how the growth rates and xylose incorporation rates varied depending on various types of medium composition when the recombinant yeast was cultured in media containing xylose. It can be understood based on Non-Patent Document 1 that if an amino acid mixture is added to a minimal medium (YNB medium) for yeast, the xylose incorporation rate is not improved (see Table 2 in Non-Patent Document 1).

In addition, Patent Document 3 discloses a technology of using as an additive a component containing a fermentation product after the culture of a filamentous fungi in a bran medium during a simultaneous saccharification-fermentation process using a carbohydrate raw material (and in particular, starch material). Also, the document discloses that such an additive may be composed of amino acids such as alanine, arginine, asparagine, aspartic acid, valine, leucine, and glutamic acid. However, with the technology disclosed in Patent Document 3, the principal component of an additive is the above-mentioned fermentation product. Hence, the document does not disclose a technology such that an amino acid is independently used as an additive. Also, the document completely differs from a technology of improving the xylose incorporation rate of yeast having xylose-metabolizing ability.

### Patent Documents

Patent Document 1: JP Patent Publication (Kohyo) No. 2000-509988 A
Patent Document 2: JP Patent Publication (Kohyo) No. 2008-506383 A
Patent Document 3: JP Patent Publication (Kohyo) No. 2009-529903 A

### Non-Patent Document

Non-Patent Document 1: Barbel Hahn-Hagerdal et al., Microbial Cell Factories 2005, 4: 31.

### SUMMARY OF THE INVENTION

As described above, attempts have been made to improve the xylose-metabolizing ability, and particularly the xylose incorporation rate of yeast to which xylose-metabolizing ability has been imparted, but currently none of them have achieved sufficient effects. Hence, an object of the present invention is to provide a fermentation culture method by which the xylose-metabolizing ability and particularly the xylose incorporation rate of yeast to which xylose-metabolizing ability has been imparted can be significantly improved.

As a result of intensive studies to achieve the above object, the present inventors have found that the xylose incorporation rate of yeast to which xylose-metabolizing ability has been imparted can be improved in a medium containing a predetermined amino acid, and thus have completed the present invention. Specifically, the present invention encompasses the following (1) to (7).

(1) A fermentation culture method, comprising the steps of: culturing yeast having xylose-metabolizing ability in a xylose-containing medium in which the concentration of at least one amino acid selected from the group consisting of asparagine (Asn), serine (Ser), tyrosine (Tyr), threonine (Thr), and histidine (His) is increased; and recovering alcohol from the medium.
(2) The fermentation culture method according to (1), wherein the xylose-containing medium is prepared by adding a solution containing the above amino acid(s) to a basal medium containing xylose.
(3) The fermentation culture method according to (2), wherein the basal medium contains saccharified woody biomass.
(4) The fermentation culture method according to (1), wherein the culture of yeast is initiated in a xylose-containing medium having the increased concentration of the amino acid(s).
(5) The fermentation culture method according to (1), wherein a solution containing the amino acid(s) is added during a step of culturing the yeast in a basal medium containing xylose.
(6) The fermentation culture method according to (1), wherein the yeast is recombinant yeast prepared by introducing a xylose-metabolization-related gene group into Saccharomyces cerevisiae.
(7) The fermentation culture method according to (6), wherein a β-glucosidase gene is further introduced into the recombinant yeast.

The fermentation culture method according to the present invention can significantly improve the xylose-metabolizing ability of yeast having xylose-metabolizing ability. Therefore, according to the fermentation culture method of the present invention, the efficiency of alcohol production using xylose as a substrate can be significantly improved with the use of yeast having xylose-metabolizing ability.

### BRIEF DESCRIPTION OF THE DRAWINGS

FIG. 1 is a block diagram schematically showing pXDI-HOR7p-ScTAL1-ScTKL1.

FIG. 2 is a block diagram schematically showing pXEL-HOR7p-ScRPE1-ScRKI 1.

FIG. 3 is a block diagram schematically showing pXIH-HOR7p-XKS1.

FIG. 4 is a block diagram schematically showing pRS524-HOR7p-PiXI.

FIG. 5 shows the analytical results of real-time quantitative PCR. FIG. 5 is a characteristic diagram showing the results of a relative comparison of the gene expression levels of strains prepared in Examples.

FIG. 6 is a block diagram schematically showing pXLG-HOR7p-BGL1.

FIG. 7 is a characteristic diagram showing the results of comparing the amounts of xylose incorporated when various amino acids were added to media containing xylose.

FIG. 8 is a characteristic diagram showing the results of comparing the decreased amounts of glucose when various amino acids were added to media containing xylose.

FIG. 9 is a characteristic diagram showing the results of comparing the growth rates when various amino acids were added to media containing xylose.

### DESCRIPTION OF THE PREFERRED EMBODIMENTS

The fermentation culture method according to the present invention is a method for fermentation production of alcohol or the like by culturing yeast having xylose-metabolizing ability in a xylose-containing medium, so as to improve the xylose-metabolizing ability of the yeast. Here, the term “xylose-metabolizing ability” refers to the efficiency of a fermentation reaction by which xylose contained in a medium is metabolized to generate alcohol. Therefore, improvement of xylose-metabolizing ability is synonymous with improvement of reaction efficiency in the fermentation reaction. The xylose-metabolizing ability of yeast can be evaluated using the rate of incorporation of xylose contained in a medium as an indicator, for example. Xylose incorporation rate can be calculated by measuring over time the decreased amount of xylose having a known concentration at the beginning of culture. Here, xylose-metabolizing ability can also be evaluated by quantitatively determining alcohol produced using xylose as a substrate.

### Yeast Having Xylose-Metabolizing Ability

In the fermentation culture method according to the present invention, yeast having xylose-metabolizing ability is used. Yeast to be used in the present invention may be yeast inherently having xylose-metabolizing ability or yeast to which xylose-metabolizing ability has been imparted or in which it has been enhanced via introduction of a given gene. Examples of yeast inherently having xylose-metabolizing ability include, but are not particularly limited to, Candida shehatae, Pachysolen tannophilius, and Pichia stipitis.

Also, a xylose-metabolization-related gene is introduced into yeast inherently lacking xylose-metabolizing ability, so that xylose-metabolizing ability can be imparted to the yeast. The term “xylose-metabolization-related gene” is meant to include a xylose reductase gene encoding xylose reductase that converts xylose to xylitol, a xylitol dehydrogenase gene encoding xylitol dehydrogenase that converts xylitol to xylulose, a xylose isomerase gene encoding xylose isomerase that converts xylose to xylulose, and a xylulokinase gene encoding xylulokinase that phosphorylates xylulose to generate xylulose 5-phosphate. In addition, xylulose 5-phosphate generated by xylulokinase enters the pentose phosphate pathway so as to be metabolized.

Examples of a xylose-metabolization-related gene to be introduced into yeast include, but are not particularly limited to, a Pichia stipitis-derived xylose reductase gene and xylitol dehydrogenase gene, and a Saccharomyces cerevisiae-derived xylulokinase gene (see Eliasson A. et al., Appl. Environ. Microbiol, 66: 3381-3386; and Toivari M N et al., Metab. Eng. 3: 236-249). In addition to these examples, as a xylose reductase gene, a Candida tropicalis- or Candida prapsilosis-derived xylose reductase gene can be used. As a xylitol dehydrogenase gene, a Candida tropicalis- or Candida prapsilosis-derived xylitol dehydrogenase gene can be used. As a xylulokinase gene, a Pichia stipitis-derived xylulokinase gene can be used. In addition, a Streptomyces murinas cluster- or Piromyces-derived xylose isomerase gene can be used, for example.

Examples of yeast that can be used for introduction of a xylose-metabolization-related gene include, but are not particularly limited to, ascomycetous yeast of Ascomycetes (Ascomycota), basidiomycetous yeast of Basidiomycetes (Basidiomycota), and yeast of Deuteromycetes (Fungi Imperfecti). Preferably, ascomycetous yeast, particularly budding yeast such as Saccharomyces cerevisae, Candida utilis, or Pichia pastris, and fission yeast such as Shizosaccharomyces pombe, are used, for example. Also, specific examples of yeast include Saccharomyces cerevisae A451, YPH499, YPH500, W303-1A, and W303-1B strains.

Furthermore, yeast having xylose-metabolizing ability, which can be preferably used herein is yeast exhibiting β glucosidase activity or xylan-degrading activity, into which a β glucosidase gene or a xylan-degrading enzyme gene has been introduced so that the genes can be expressed. The β glucosidase gene or the xylan-degrading enzyme gene may be introduced into yeast in a form having a signal sequence so that the β glucosidase or the xylan-degrading enzyme is secreted outside the microorganism. Alternatively, the gene may be introduced into yeast in a form fused to a cell surface layer localized protein gene so that the β glucosidase or the xylan-degrading enzyme is displayed on the cell surface layer.

In addition, a method for introducing the β glucosidase gene or the xylan-degrading enzyme gene, so that the β glucosidase or the xylan-degrading enzyme is displayed on the cell surface layer is not particularly limited, and a technique described in WO 2010-005044 can be employed, for example. Also, examples of the β glucosidase gene include, but are not particularly limited to, Aspergillus aculeatus-derived β glucosidase gene (Murai et al., Appl. Environ. Microbiol. 64: 4857-4861). In addition to such an example, as the βglucosidase gene, Aspergillus oryzae-derived β glucosidase gene, Clostridium cellulovorans-derived β glucosidase gene, or Saccharomycopsis fibligera-derived β glucosidase gene can be used, for example.

Also, as a method for introducing the above xylose-metabolization-related gene or a cell surface display-type β glucosidase gene and the xylan-degrading enzyme gene, any technique conventionally known as a yeast transformation method can be applied. Specifically, introduction of the genes can be achieved by electroporation (described in Meth. Enzym., 194, p 182 (1990)), a spheroplast method (described in Proc. Natl. Acad. Sci. U.S.A., 75 p 1929 (1978), or a lithium acetate method (described in J. Bacteriology, 153, p 163 (1983); Proc. Natl. Acad. Sci. U.S.A., 75 p 1929 (1978); Methods in yeast genetics, 2000 Edition: A Cold Spring Harbor Laboratory Course Manual), for example. However, the examples of the method are not limited to them.

Also, when these genes are introduced, plasmid DNA that is used for yeast transformation can be used. Examples of such a plasmid DNA include an YCp-type Escherichia coli-yeast shuttle vector such as pRS413, pRS414, pRS415, pRS416, YCp50, pAUR112, or pAUR123, an YEp-type Escherichia coli-yeast shuttle vector such as pYES2 or YEp13, a YIp-type Escherichia coli-yeast shuttle vector such as pRS403, pRS404, pRS405, pRS406, pAUR101, or pAUR135, an Escherichia coli-derived plasmid (e.g., a ColE plasmid such as pBR322, pBR325, pUC18, pUC19, pUC118, pUC119, pTV118N, pTV119N, pBluescript, pHSG298, pHSG396, or pTrc99A, a p 15A plasmid such as pACYC177 or pACYC184, or a pSC101 plasmid such as pMW118, pMW119, pMW218, or pMW219), Bacillus subtilis-derived plasmid (e.g., pUB110 or pTP5). Examples of phage DNA include λ phages (e.g., Charon4A, Charon21A, EMBL3, EMBL4, λgt10, λgt11, and λZAP), φX174, M13 mp 18, or M13 mp 19. An example of retrotransposon is a Ty factor. An example of a YAC vector is pYACC2.

Furthermore, when these genes are introduced, a conventionally known selection marker gene, a transcription promoter such as a constant expression-type promoter or an induced expression-type promoter, a cis element such as an enhancer, splicing signals, polyA addition signals, a transcription terminator, and the like can be appropriately used.

### <Ethanol Production>

Ethanol fermentation using sugar such as xylose as a substrate can be performed through the use of the above explained yeast having xylose-metabolizing ability. Particularly in the fermentation culture method according to the present invention, the xylose-metabolizing ability of yeast having xylose-metabolizing ability is improved with the use of a specific amino acid(s). Here, the term “specific amino acid” refers to at least one amino acid selected from the group consisting of asparagine (Asn), serine (Ser), tyrosine (Tyr), threonine (Thr), and histidine (His).

In the fermentation culture method according to the present invention, the above yeast is cultured in a medium in which the concentration of the above specific amino acid(s) is increased. The term “a medium in which the concentration of the above specific amino acid(s) is increased” refers to a medium prepared by adding the amino acid to a basal medium. The term “basal medium” is meant to include a medium that is conventionally used for culturing yeast, a solution obtained by subjecting woody biomass to saccharification, and a mixture thereof. Such a basal medium may have composition containing various amino acid components including the above specific amino acid(s) or composition not containing such amino acid component. A medium, which is obtained by adding the amino acid to a medium containing the above specific amino acid(s) is also included in examples of the medium in which the concentration of a specific amino acid(s) is increased.

Examples of a medium that is conventionally used for culturing yeast include, but are not limited to, an SD medium, an YPD medium, an YPAD medium, an YM medium, and various synthetic media containing Yeast Nitrogen Base. Also, examples of a technique for saccharification of woody biomass are not particularly limited and any conventionally known technique can be used without particular limitation. Examples of a saccharification method include a sulfuric acid method using dilute sulfuric acid or concentrated sulfuric acid, and an enzyme method using cellulase or hemicellulose. Also, prior to saccharification, woody biomass may be subjected to conventionally known pretreatment. Examples of pretreatment include, but are not particularly limited to, treatment of degrading lignin by microorganisms and treatment of crushing or grinding woody biomass.

In particular, in the fermentation culture method according to the present invention, xylose-metabolizing ability of the above yeast is improved by addition of the above specific amino acid(s) and a medium to be used herein contains xylose. However, the concentration of xylose in a medium preferably ranges from 0 g/l to 800 g/l, more preferably ranges from 0 g/l to 400 g/l, and further preferably ranges from 0 g/l to 200 g/l, but the examples are not particularly limited to these ranges.

Also, the above specific amino acid(s) may be added to a medium at the beginning of the culture of the yeast or may be added at a desired time during the culture process of the above yeast. For example, at the initial stage of culture, the above specific amino acid(s) is not added to a medium, hexose such as glucose contained in the medium is mainly metabolized by the above yeast, and then the above specific amino acid(s) is added at a predetermined time to the medium, so that the rate of metabolization of xylose contained in the medium can be improved.

In addition, upon the above ethanol fermentation using yeast, the above yeast is preferably cultured under optimum conditions. Preferable culture conditions for the above yeast comprise temperatures ranging from 25° C. to 35° C. and pH levels ranging from 4 to 6. Also, upon culture, agitation or shaking may be performed.

Also, in the fermentation culture method according to the present invention, ethanol resulting from fermentation production from a carbon source such as xylose contained in a medium is recovered. Examples of a method for recovering ethanol are not particularly limited, and any conventionally known method can be applied. For example, after completion of the above ethanol fermentation, a liquid layer containing ethanol is separated from a solid layer containing recombinant yeast and solid components by solid-liquid separation. Subsequently, ethanol contained in the liquid layer is separated and purified by a distillation method, so that ethanol with high purity can be recovered. In addition, the purification degree of ethanol can be appropriately adjusted depending on the purpose of use of ethanol.

According to the fermentation culture method of the present invention, the xylose-metabolizing ability of yeast can be significantly improved, and thus ethanol productivity can be improved in ethanol fermentation using a medium containing xylose. Therefore, when the fermentation culture method according to the present invention is applied to a system for producing ethanol from woody biomass; for example, alcohol yield per unit amount of biomass can be improved.

### EXAMPLES

The present invention will be specifically described in the following examples. However, the examples are not intended to limit the technical scope of the present invention.

### Example 1

### Yeast Having Xylose-Metabolizing Ability

First, in Example 1, yeast having xylose-metabolizing ability was prepared using the Saccharomyces cerevisiae W303-1B strain (ATCC number: 201238). An outline is provided as follows. Expression of the 4 genes (TAL1, TKL1, RPE1, and RKI1) of the pentose phosphate pathway of the W303-1B strain and a xylulose kinase gene, XKS1, was enhanced. An aldose reductase gene, GRE3, was disrupted, multiple copies of a Piromyces sp. E2 strain-derived xylose isomerase (XI) gene were introduced onto a chromosome, and then a recombinant yeast W801M strain was prepared through introduction enabling secretory production of Aspergillus aculeatus-derived β-glucosidase BGL1.

Specifically, vectors for introduction of a TAL1 gene, a TKL1 gene, a RPE1 gene, a RKI1 gene, a XKS1 gene, and a PiXI gene were constructed as described below.

### (1) Vector for Introduction of TAL1 and TKL1 Genes

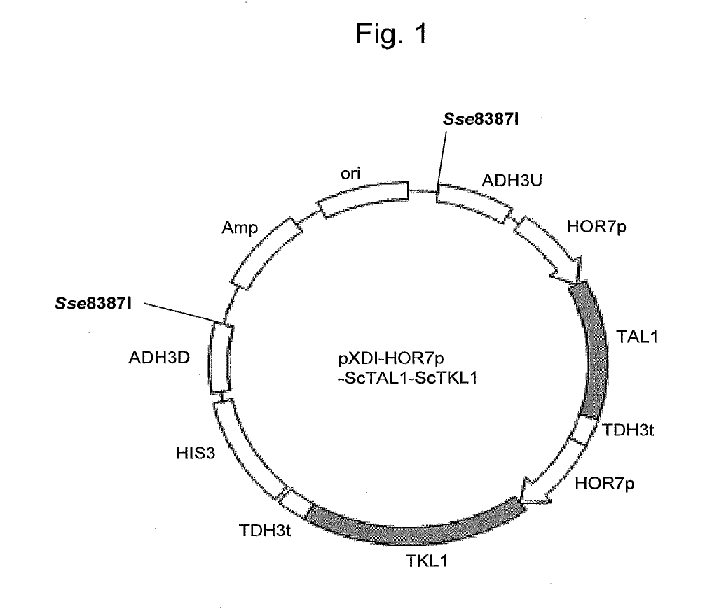

A pXDI-HOR7p-ScTAL1-ScTKL1 vector was constructed for yeast introduction, to be used for an S. cerevisiae-derived transaldolase (TAL1) gene and a transketolase 1 (TKL1) gene (FIG. 1). The vector contained an S. cerevisiae S288 strain-derived TAL1 gene (GenBank Accession: X15953) with an HOR7 promoter added to the 5′ side and a TDH3 terminator added to the 3′ side. Also, the vector contained S. cerevisiae S288 strain-derived TKL1 gene (GenBank: X73224) containing an HOR7 promoter added to the 5′ side and a TDH3 terminator added to the 3′ side. Furthermore, the vector contained, as regions for homologous recombination into the yeast genome, the DNA sequence (ADH3U) of a roughly 500-bp non-coding region upstream of an alcohol dehydrogenase3 (ADH3) gene, the DNA sequence (ADH3D) of a roughly 500-bp non-coding region downstream of the ADH3 gene, and as a marker, a gene sequence (HIS3) containing the HIS3 histidine synthase gene.

### (2) Vector for Introduction of RPE1 and RKI1 Genes

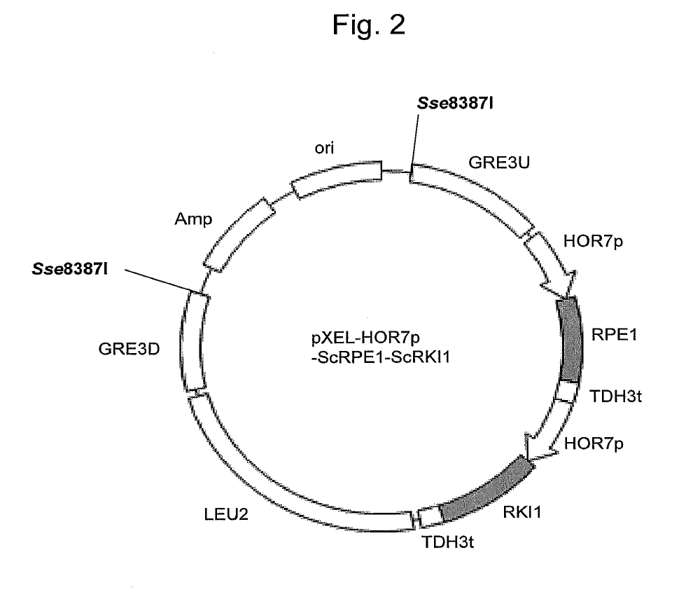

A pXEL-HOR7p-ScRPE1-ScRKI1 vector was constructed for introduction of S. cerevisiae-derived ribulosephosphate epimerase (RPE1) gene and ribosephosphate ketoisomerase (RKI1) gene into yeast (FIG. 2). The vector contained S. cerevisiae S288 strain-derived RPE1 gene (GenBank: X83571) containing a HOR7 promoter added to the 5′ side and a TDH3 terminator added to the 3′ side. Also, the vector contained S. cerevisiae S288 strain-derived RKI1 gene (GenBank: Z75003) containing a HOR7 promoter added to the 5′ side and a TDH3 terminator added to the 3′ side. Furthermore, the vector contained, as regions for homologous recombination onto yeast genome and disruption of an aldose reductase (GRE3) gene, an about 1000-bp gene sequence (GRE3D) upstream of a GRE3 gene, the gene sequence (GRE3D) of a roughly 800-bp region including about 500-bp 3′ region of the GRE3 gene, and as a marker, a gene sequence (LEU2) containing a leucine synthase gene, LEU2.

### (3) Vector for Introduction of XKS1 Gene

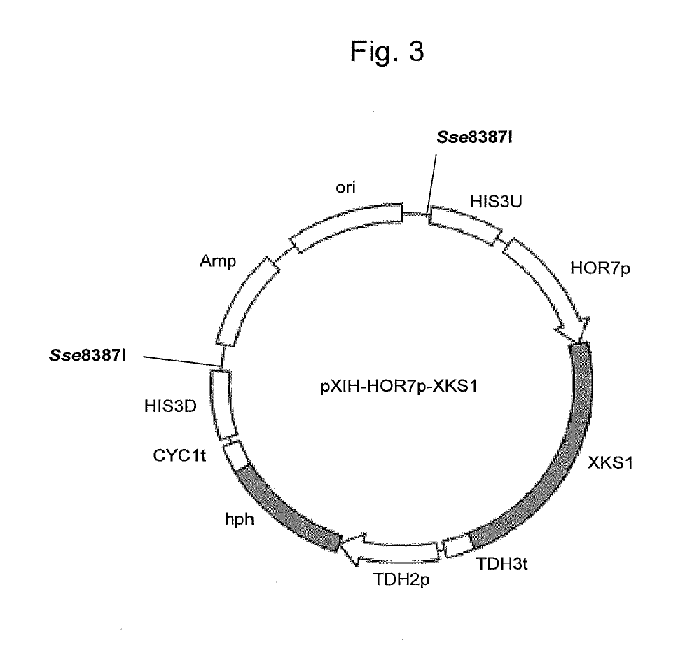

A pXIH-HOR7p-XKS1 vector was constructed for introduction of the yeast S. cerevisiae-derived xylulokinase (XKS1) gene into yeast (FIG. 3). The vector contained S. cerevisiae S288 strain-derived XKS1 gene (GeneBank: Z72979) containing an HOR7 promoter added to the 5′ side and a TDH3 terminator added to the 3′ side. Furthermore, the vector contained, as regions for homologous recombination onto yeast genome, the gene sequence (HIS3U) of an about 500-bp region upstream of a histidine synthase (HIS3) gene, the gene sequence (HIS3D) of an about 500-bp region downstream of the HIS3 gene, and as a marker, a hygromycin phosphotransferase (hph) gene (GeneBank: V01499) containing a TDH2 promoter added to the 5′ side and a CYC1 terminator added to the 3′ side.

### (4) Vector for Introduction of PiXI Gene (Multicopy Integration)

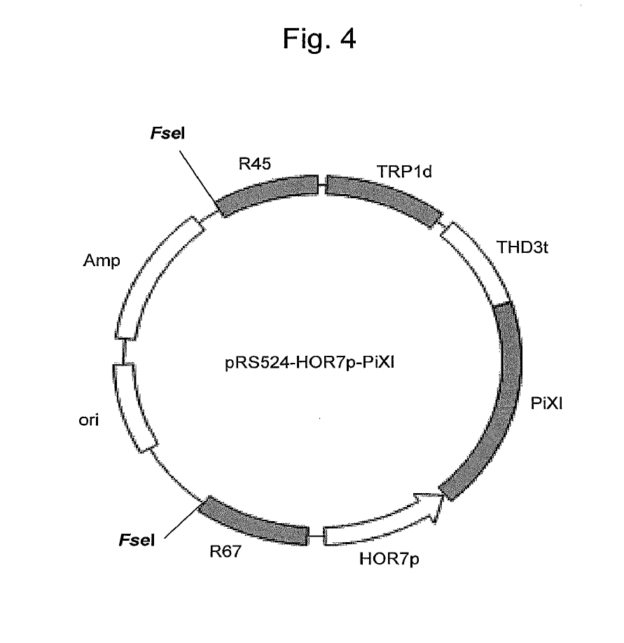

A pRS524-HOR7p-PiXI vector that enables multicopy integration of Piromyces sp. E2-derived XI gene (PiXI) onto a chromosome was constructed (FIG. 4). The vector contained the PiXI gene (GenBank: AJ249909) containing an HOR7 promoter added to the 5′ side and a TDH3 terminator added to the 3′ side. Furthermore, the vector contained, as regions for homologous recombination onto yeast genome, R45 and R67 gene sequences homologous to the rRNA gene (rDNA), and as a marker, the gene sequence of a TRP1d marker having the expression level lowered via deletion of a promoter portion. These R45 and R67 gene sequences enable multicopy integration of the gene sequence containing PiXI to the rDNA locus on chromosome 12. Furthermore, the TRP1d marker functions as a marker only in the case of multicopy integration thereof onto a chromosome. Hence, transformants in which multicopies of the gene sequence containing PiXI have been introduced can be selected.

### (5) Preparation of Yeast Strains Capable of Assimilating Xylose

Yeast strains capable of assimilating xylose were prepared using vectors prepared in (1) to (4) above, respectively. Yeast was transformed according to attached protocols using Frozen-EZ Yeast Transformation II (ZYMO RESEARCH). Variations of medium composition employed herein are listed in Table 1.

TABLE 1

| Medium name | Medium composition |
|---|---|
| SD medium | 6.7 g/l Yeast Nitrogen Base without amino acids, 20 g/l |
| | D-Glucose |
| SX medium | 6.7 g/l Yeast Nitrogen Base without amino acids, 20 g/l |
| | D-Xylose |
| SD + all medium | Add 50x amino acid mixture (all) to SD medium in a |
| | volume 1/50 the volume of the medium. |
| SD-His medium | Add 50x amino acid mixture (-His) to SD medium in a |
| | volume 1/50 the volume of the medium. |
| SD-HL medium | Add 50x amino acid mixture (-HL) to SD medium in a |
| | volume 1/50 the volume of the medium. |
| SX + all medium | Add 50x amino acid mixture (all) to SX medium in a |
| | volume 1/50 the volume of the medium. |
| SX-His medium | Add 50x amino acid mixture (-His) to SX medium in a |
| | volume 1/50 the volume of the medium. |
| SX-HL medium | Add 50x amino acid mixture (-HL) to SX medium in a |
| | volume 1/50 the volume of the medium. |
| SD agar medium | SD medium + 20 g/l Agar |
| SD-His agar medium | Add 50x amino acid mixture (-His) to SD agar medium in |
| | a volume 1/50 the volume of the medium. |
| SD-HL agar medium | Add 50x amino acid mixture (-HL) to SD agar medium in |
| | a volume 1/50 the volume of the medium. |
| 50x amino acid mixture (all) | 1 g/l L-adenine sulfate, 1 g/l L-histidine, 5 g/l L-leucine, 1 |
| | g/l L-tryptophan, 1 g/l Uracil |
| 50x amino acid mixture (-His) | 1 g/l L-adenine sulfate, 5 g/l L-leucine, 1 g/l |
| | L-tryptophan, 1 g/l Uracil |
| 50x amino acid mixture (-HL) | 1 g/l L-adenine sulfate, 1 g/l L-tryptophan, 1 g/l Uracil |
| YPX5 medium | 10 g/l Yeast Extract, 20 g/l Polypeptone, 50 g/l Xylose |

First, the full-length ADE2 gene (GenBank: M59824) was amplified by PCR using an S. cerevisiae S288 strain-derived genome DNA as a template. A yeast S. cerevisiae W303-1B strain (Matα ade2 his3 leu2 trp1 ura3) was transformed using the amplification product and then SD-A agar medium was coated with the resultant. Colonies that had grown were subjected to streak culture on new SD-A agar medium, so that they were thus purified. The thus selected strains for which the adenine requirement had been satisfied as a result of purification were designated W303-1BA strains. For PCR amplification of the ADE2 gene, ADE2+1F (5′-atggattctagaacagttggtatattagg-3′: SEQ ID NO: 1) and ADE2+1716R (5′-ttacttgttttctagataagcttcgtaacc-3′: SEQ ID NO: 2) primers were used.

Next, a fragment was obtained by digestion of about 1 μg of the pXDI-HOR7p-ScTAL1-ScTKL1 vector shown in FIG. 1 with an Sse8387I restriction enzyme. An S. cerevisiae W303-1BA strain as host yeast was transformed using the fragment, SD-AH agar medium was coated with the resultant, and then colonies that had grown were subjected to streak culture on new SD-AH agar medium to purify the colonies. The thus purified selected strain was designated a W200 strain.

Next, a fragment was obtained by digesting about 1 μg of the pXEL-HOR7p-ScRPE1-ScRKI1 vector shown in FIG. 2 with a Sse8387I restriction enzyme. The W200 strain was transformed using the fragment, an SD-AHL agar medium was coated with the resultant, and then colonies that had grown were subjected to streak culture on a new SD-AHL agar medium to purify the colonies. The thus purified selected strain was designated W500.

Next, a fragment was obtained by digesting about 1 μg of the pXIH-HOR7p-XKS1 vector shown in FIG. 3 using a Sse8387I restriction enzyme. The W500 strain was transformed using the fragment, an YPD agar medium (YPD+hyg) containing 150 μg/ml hygromycin B (Wako Pure Chemical Industries, Ltd.) was coated with the resultant, and then colonies that had grown were subjected to streak culture on a new YPD+hyg agar medium to purify the colonies. The thus purified selected strain was designated a W600 strain.

Next, a fragment was obtained by digesting about 1 μg of the pRS524-HOR7p-PiXI shown in FIG. 4 with a restriction enzyme Fse I. The W600 strain was transformed using the fragment, an SD-AHLW agar medium was coated with the resultant, and then colonies that had grown were subjected to streak culture on a new SD-AHLW agar medium to purify the colonies. The thus purified selected strain was designated a W700M2 strain. Furthermore, an SX-AHLW agar medium containing xylose as a carbon source was coated with the W700M2 strain, followed by 48 hours of culture at 30° C. The growth of microorganisms could be confirmed.

### (6) Confirmation of the Expression Levels of Introduced Genes

The expression levels of introduced genes in the thus prepared yeast strains were quantitatively determined and then compared by the following method. Host strains, the W303-1B strain, the W200 strain, the W500 strain, the W600 strain, and the W700M2 strain, were cultured in SD+all medium, SD-AH medium, SD-AHL medium, SD-AHL medium, and SD-AHLW medium (5 ml each), respectively, at 30° C. for 24 hours. Microorganisms were collected, washed with sterile water, and then suspended in sterile water to a concentration (OD600) of 10. The suspension (600 μl each) was dispensed into Eppendorf tubes, and then centrifuged at 500×g for 2 minutes, so as to remove the supernatants. Total RNA was extracted from the thus collected microorganisms using a YeaSter RNA Kit (ZYMO RESEARCH). DNA degradation and total RNA purification were performed using a DNA-Free RNA Kit (ZYMO RESEARCH) in order to remove the remaining DNA from the extracts. The thus purified total RNA solutions were stored in a deep freezer at −80° C. until reverse transcription reaction.

The concentration of total RNA in each sample was measured, and then reverse transcription reaction from total RNA was performed using High-Capacity cDNA Reverse Transcription Kits (Applied Biosystems). The amount of total RNA used herein was 0.2 μg, and the remainders in the reaction solution composition were determined according to attached protocols. Reverse transcription reaction was performed using GeneAmp PCR system 9700 (Applied Biosystems). The reaction time and temperature were determined according to the attached protocols and then the reaction was performed. Samples after the reaction were stored at −20° C. until use. Genes to be quantitatively determined were the PiXI gene, the XKS1 gene, the TAL1 gene, the TKL1 gene, the RPE1 gene, the RKI1 gene, and the GRE3 gene. TUB1 and UBC6 were selected as control house keeping genes. Power SYBR Green PCR Master Mix (Applied Biosystems) was used as a reaction reagent for Real-time quantitative PCR. The reaction composition was determined according to the attached protocols. Primer sequences used herein are together shown in Table

TABLE 2

| Primer | Sequence | | Length |
|---|---|---|---|
| PiXI-F | 5′-tacgctcgttccaagggatt-3′ | (SEQ ID NO: 3) | 20 |
| PiXI-R | 5′-tggtgcttggttggttccat-3′ | (SEQ ID NO: 4) | 20 |
| XKS1-F | 5′-cagcagcacgggtctgtcta-3′ | (SEQ ID NO: 5) | 20 |
| XKS1-R | 5′-cggtttcttattcaattgctctaaca-3′ | (SEQ ID NO: 6) | 26 |
| TAL1-F | 5′-tgccaagcaaccaacttacg-3′ | (SEQ ID NO: 7) | 20 |
| TAL1-R | 5′-cggtggtcttaccatgcttc-3′ | (SEQ ID NO: 8) | 20 |
| TKL1-F | 5′-gcgcatgaacccaacca-3′ | (SEQ ID NO: 9) | 17 |
| TKL1-R | 5′-cgaccgcgtgaccgtta-3′ | (SEQ ID NO: 10) | 17 |
| RPE1-F | 5′-gacatgatgccaaaagtggaaa-3′ | (SEQ ID NO: 11) | 22 |
| RPE1-R | 5′-ccaccatcgacttggatattca-3′ | (SEQ ID NO: 12) | 22 |
| RKI1-F | 5′-tgccggtgtcccaaaaa-3′ | (SEQ ID NO: 13) | 17 |
| RKI1-R | 5′-tgcagctctcttggcatcc-3′ | (SEQ ID NO: 14) | 19 |
| GRE3-F | 5′-ggcgcagatgacgagaaga-3′ | (SEQ ID NO: 15) | 19 |
| GRE3-R | 5′-cagagcccggtacgtatctatga-3′ | (SEQ ID NO: 16) | 23 |
| TUB1 F | 5′-ccaactggtttcaagatcggta-3′ | (SEQ ID NO: 17) | 22 |
| TUB1 R | 5′-tccacagtggccaattgtga-3′ | (SEQ ID NO: 18) | 20 |
| UBC6 F | 5′-cggcaaatacaggtgatgaaac-3′ | (SEQ ID NO: 19) | 22 |
| UBC6 R | 5′-tcctccaacgagatgactttttc-3′ | (SEQ ID NO: 20) | 23 |

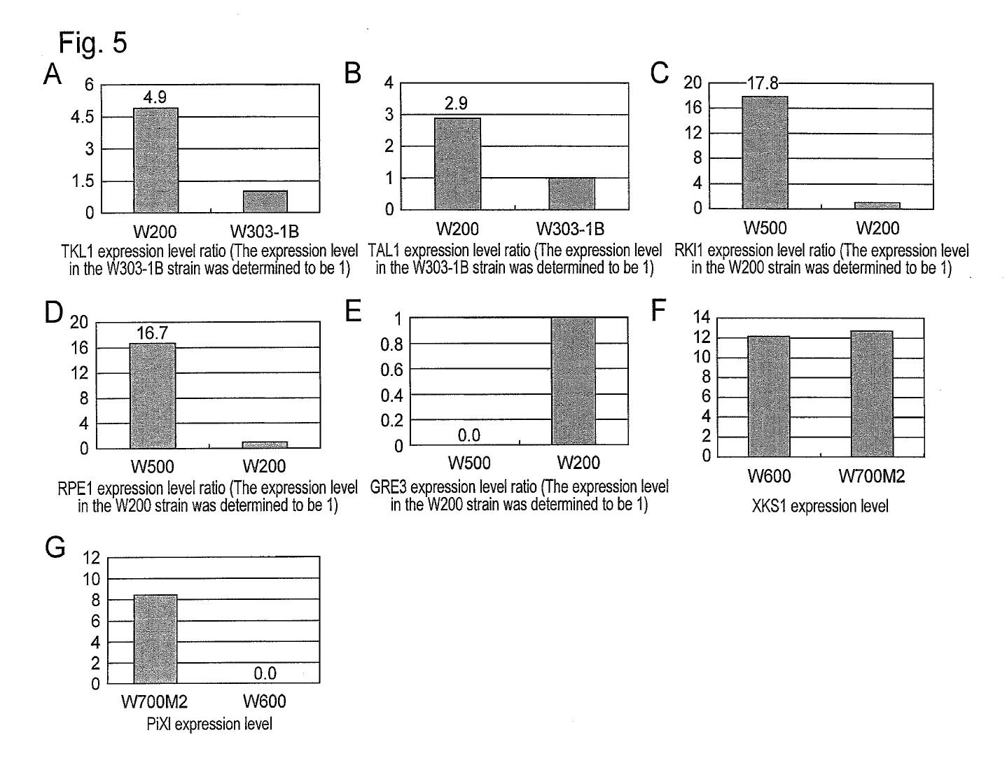

Relative comparison of gene expression levels in strains was conducted based on the analytical results of real-time quantitative PCR. FIG. 5 shows the results. At this time, sample-to-sample expression level ratios were corrected using the TUB1 gene and the UBC6 gene as internal control. As shown in FIG. 5A and FIG. 5B, it was confirmed that in the W200 strain, the TKL1 gene and the TAL1 gene were expressed at levels higher than in the W303-1B strain, which was the parent strain. Next, as shown in FIG. 5C and FIG. 5D, it was confirmed that the RKI1 gene and the RPE1 gene exhibited improved expression levels in the W500 strain, compared with the W200 strain. Moreover, it was confirmed that the GRE3 gene was not expressed in the W500 strain (FIG. 5E). Next, as shown in FIG. 5F, it was confirmed that the XKS1 gene was expressed in the W600 strain and the W700M2 strain. Furthermore, as shown in FIG. 5G, it was confirmed that the PiXI gene was expressed at a high level in the W700M2 strain.

### (7) Introduction of β-Glucosidase BGL1 Gene

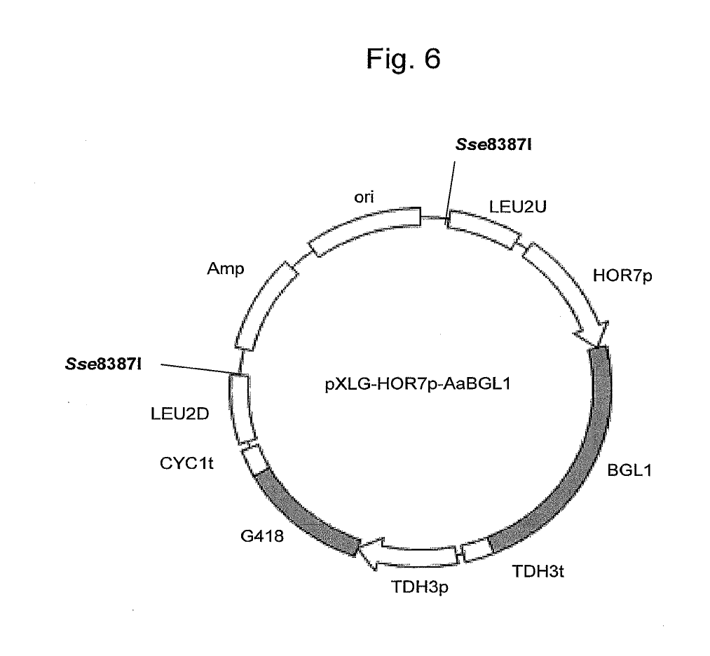

Next, a recombinant yeast W801M strain was prepared by introducing a BGL1 gene to the W700M2 strain prepared in (5) above, so as to enable secretory production of Aspergillus aculeatus-derived β-glucosidase BGL1. First, a pXLG-HOR7p-BGL1 vector for introduction of Aspergillus aculeatus-derived β-glucosidase (BGL1) gene into yeast was constructed (FIG. 6). The vector contained a gene sequence containing a sequence encoding the secretory signal of Rhizopus oryzae-derived glucoamylase (GeneBank: D00049) added to the sequence encoding the mature protein of Aspergillus aculeatus-derived BGL1 (GeneBank: D64088). Also, the vector contained an HOR7 promoter added to the 5′ side and a TDH3 terminator added to the 3′ side of the gene sequence. Furthermore, the vector contained, as regions for homologous recombination onto yeast genome, the gene sequence (HIS3U) of a roughly 500-bp region upstream of the leucine synthase (LEU2) gene, the gene sequence (LEU2D) of a roughly 500-bp region downstream of the LEU2 gene, and as a marker, a gene sequence containing the geneticin resistance gene (G418) containing a TDH3 promoter added to the 5′ side and a CYC1 terminator added to the 3′ side.

Next, a fragment was obtained by digesting about 1 μg of the pXLG-HOR7p-BGL1 vector shown in FIG. 6 with a Sse8387I restriction enzyme. The W700M2 strain was transformed using the fragment, an YPD agar medium (YPD+G418) containing 200 μg/ml Genetcin Disulfide (Wako Pure Chemical Industries, Ltd.) was coated with the resultant, and then colonies that had grown were subjected to streak culture on a new YPD+G418 agar medium to purify the colonies. The thus purified selected strain was designated a W801M strain.

(8) Simultaneous (Glucose.Xylose) Fermentation Test in Medium Supplemented with Amino Acid

The W801M strain prepared as described above was cultured in 5 ml of an YNG+U (6.7 g/l Yeast Nitrogen Base without amino acids, 20 g/l glucose, 20 mg/l uracil) liquid medium prepared in a test tube at 30° C. and 80 rpm for 24 hours, thereby preparing a pre-pre-culture solution. Next, the pre-pre-culture solution (5 ml) was added to new YNX+U liquid medium (250 ml) prepared in a baffled Erlenmeyer flask, followed by pre-culture at 30° C. and 100 rpm. After 48 hours of culture, the culture solution was subjected to 5 minutes of centrifugation at 1000×g at room temperature, thereby removing the supernatant.

To prevent the introduction of the substrate into the pre-culture solution, the thus collected microbial pellet was suspended in sterile water and then subjected to centrifugation at 1000×g for 5 minutes, thereby collecting microorganisms. Washing was performed twice. After washing, microorganisms were suspended in sterile water, so that a microbial solution was prepared. A sodium acetate buffer was added to the fermentation medium so as to suppress a pH decrease in the medium associated with fermentation. The microbial solution was added to an YNGXAc+U medium (6.7 g/l Yeast Nitrogen Base without amino acids, 10 g/l D-Xylose, 10 g/l Glucose, 25 mM sodium acetate, 20 mg/l uracil, pH 5.0) so that the initial microbial concentration was OD600=10. One type of amino acid was added to the YNGXAc+U medium to result in a predetermined concentration, and then the medium was used. Amino acid was dissolved in water to a concentration 10 times greater than the final concentration. Then the solution was adjusted to pH 5.0, diluted, and then used. The fermentation test was conducted as follows. Five (5) ml of the culture solution was added to a 15-ml plastic tube with a screw cap, the tube was sealed with the cap, and then the tube was shaken at 30° C. (100 rpm). Sampling from the culture solution was performed over time and thus substrates (xylose and glucose) and the product (ethanol) were analyzed via liquid chromatography. Liquid chromatography was performed using a Shim-pack SPR-Pb column (Shimadzu Corporation) at 80° C. An RID-10A refractive index detector (Shimadzu Corporation) was used. Water was used for a mobile phase and sent at a flow rate of 0.8 ml/min.

### (9) Primary Screening

A fermentation test was conducted (n=2) using as a control an YNGXAc+U medium to which nothing had been added, an YNGXAc+U medium supplemented with casamino acid (CAA, 20 g/L) (that is, an amino acid mixture obtained via hydrolysis of casein) and YNGXAc+U medium supplemented with 18 types of amino acids considered to be contained in casamino acid (20 g/L) or 2 types of amino acid (glutamine and asparagine) (5 g/L) considered not to be contained in casamino acid. The results of the measurement of the decreased amounts of xylose at hour 8 after the start of the fermentation test are shown in Table 3.

TABLE 3
Effects of addition of amino acid

| | Amount of amino | ΔXylose | Control |
|---|---|---|---|
| Amino acid added | acid added (g/l) | 0-8 h (g/l) | ratio % |
| Tyr | 0.84 | 3.62 | 136 |
| Asn | 5.00 | 3.60 | 135 |
| Ser | 1.12 | 3.42 | 128 |
| Thr | 1.22 | 3.29 | 124 |
| His | 0.54 | 3.26 | 122 |
| Lys | 1.50 | 3.08 | 116 |
| Trp | 0.22 | 3.07 | 115 |
| Gln | 5.00 | 3.05 | 115 |
| Gly | 0.44 | 3.03 | 114 |
| Pro | 1.98 | 2.97 | 112 |
| Ala | 0.56 | 2.95 | 111 |
| Arg | 0.72 | 2.91 | 109 |
| Cys | 0.06 | 2.81 | 106 |
| Phe | 0.92 | 2.79 | 105 |
| CAA | 20.00 | 2.72 | 102 |
| Val | 1.00 | 2.67 | 100 |
| Met | 0.54 | 2.67 | 100 |
| Control (No addition) | — | 2.66 | 100 |
| Asp | 1.26 | 2.46 | 92 |
| Glu | 4.22 | 2.25 | 85 |
| Ile | 1.12 | 2.09 | 79 |
| Leu | 1.68 | 1.12 | 42 |

As understood from Table 3, it was revealed that when casamino acid was added, the xylose consumption rate was not significantly improved. On the other hand, it was revealed that when tyrosine, asparagine, serine, threonine, and histidine from among 20 types of amino acid were added, the xylose consumption rate was improved by 20% or more compared with that of a control. In addition, 8 hours later, glucose had been completely consumed and thus was not detected in any samples.

### (10) Secondary Test

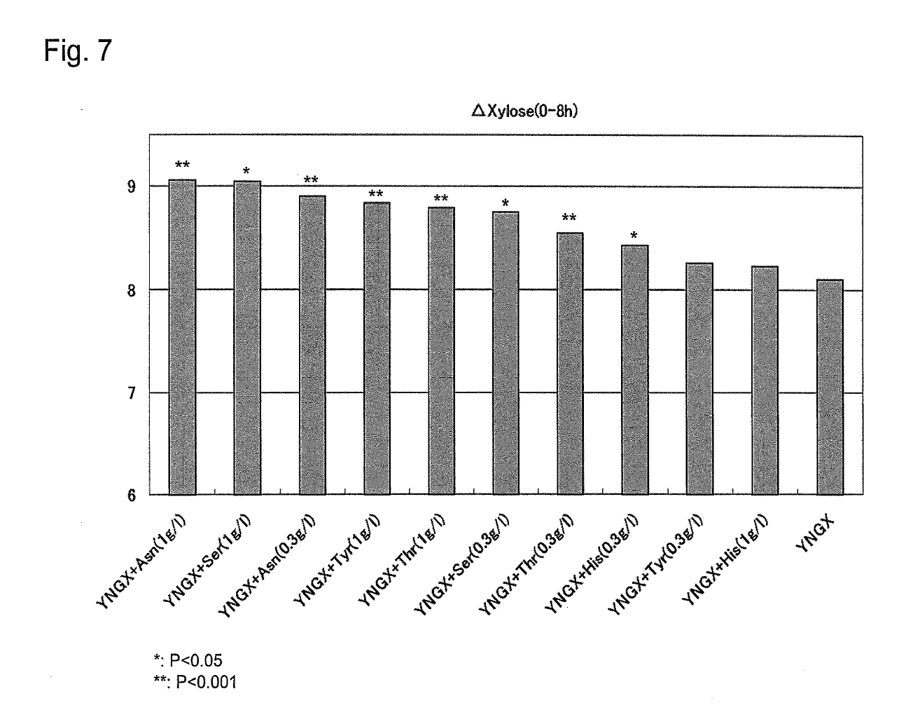

A fermentation test was conducted (n=4) by the method same as that described above for tyrosine, asparagine, serine, threonine, and histidine (the addition of which exhibited high effects in the primary screening), in which the concentration for addition was 1 g/L or 0.3 g/L. The results of measuring the decreased amounts of xylose at hour 8 after the start of the fermentation test are shown in FIG. 7. In addition, 8 hours later, glucose had been completely consumed and thus was not detected in any samples. Significant effects of improvement in xylose consumption rates compared with the control were confirmed except for the case in which 0.3 g/L tyrosine had been added and the case in which 1 g/L histidine had been added.

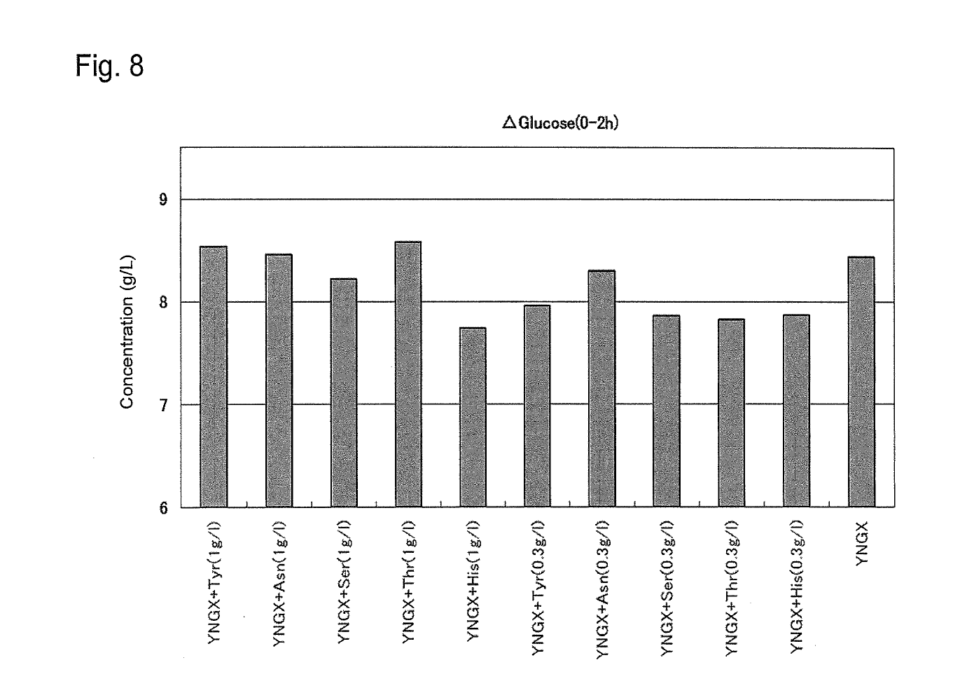

Also, 2 hours later, glucose remained in all samples. The decreased amounts of glucose at hour 2 after the start of fermentation are shown in FIG. 8. No improvement (due to the addition of the above amino acids) was observed in glucose consumption rates. It was thus revealed that xylose consumption rates can be specifically accelerated by the addition of the amino acids. Specifically, it was revealed that xylose incorporation rates can be significantly improved by adding at least one amino acid selected from among asparagine (Asn), serine (Ser), tyrosine (Tyr), threonine (Thr), and histidine (His) to a xylose-containing medium.

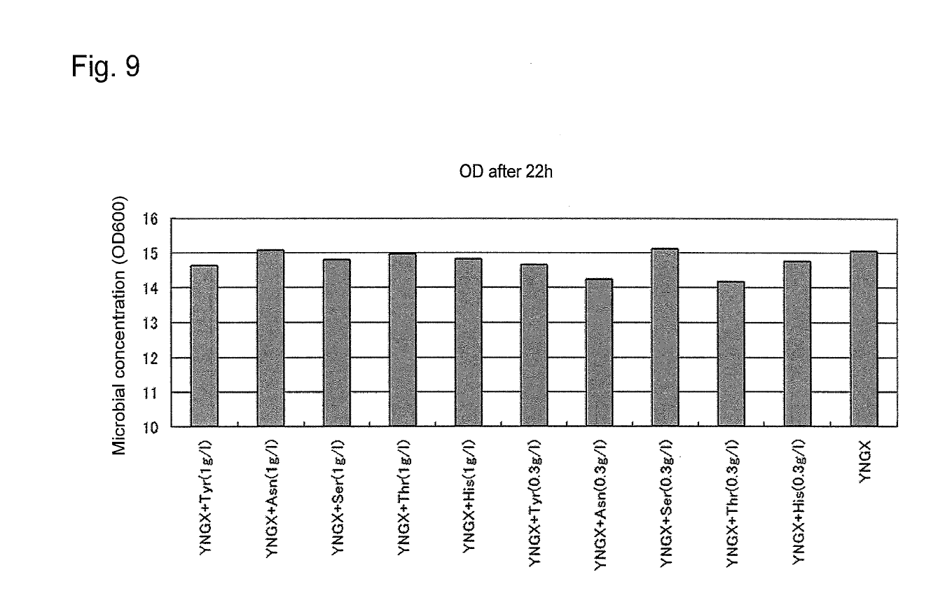

Also, 22 hours later, both glucose and xylose had been completely consumed and thus was not detected. The results of measuring the microbial concentrations (OD600) at hour 22 are shown in FIG. 9. It was understood from the results that the microbial concentrations had increased from the initial microbial concentration of OD600=10 to around OD600=15 at hour 22. However, the microbial concentrations at hour 22 when the amino acids were added were equivalent to or lower than that of the control. Hence, it was revealed that microbial growth was not accelerated by the addition of amino acids.

What is claimed is:

1. A fermentation culture method, comprising the steps of: culturing yeast having xylose-metabolizing ability in a xylose-containing medium in which the concentration of at least one amino acid selected from the group consisting of asparagine (Asn), serine (Ser), tyrosine (Tyr), threonine (Thr), and histidine (His) is increased; and recovering alcohol from the medium.

2. The fermentation culture method according to claim 1, wherein the xylose-containing medium is prepared by adding a solution containing the amino acid to a basal medium containing xylose.

3. The fermentation culture method according to claim 2, wherein the basal medium contains saccharified woody biomass.

4. The fermentation culture method according to claim 1, wherein the culture of yeast is initiated in a xylose-containing medium having the increased concentration of the amino acid.

5. The fermentation culture method according to claim 1, wherein a solution containing the amino acid is added during a step of culturing the yeast in a basal medium containing xylose.

6. The fermentation culture method according to claim 1, wherein the yeast is recombinant yeast prepared by introducing a xylose-metabolization-related gene group into Saccharomyces cerevisiae.

7. The fermentation culture method according to claim 6, wherein a β-glucosidase gene is further introduced into the recombinant yeast.
